(19) United States
(12) Patent Application Publication
(10) Pub. No.: US 2004/0255399 A1
(43) Pub. Date: Dec. 23, 2004

(54) Method and compositions for coloring hair with taurate copolymers

(75) Inventor: Jiang Yang, Edison, NJ (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/602,399

(22) Filed: Jun. 23, 2003

(51) Int. Cl. A61K 7/13

(52) U.S. Cl. 8/405

(57) ABSTRACT

A method and composition is provided for coloring hair. The method includes applying and thereafter removing from the hair a composition which includes a colorant formula incorporating an oxidation dye precursor and a developer formula incorporating an alkoxylated taurate copolymer in combination with hydrogen peroxide. The colorant and developer formulas are applied to the hair separately or together in a relative weight ratio of 10:1 to 1:10. Particularly preferred alkoxylated taurate copolymers are hydrophobically modified copolymers of acrylamidopropane sulphonic acid or salt and methacrylate esters of ethoxylated or propoxylated fatty alcohols.

### BACKGROUND OF THE INVENTION

1. Field of the Invention

The invention relates to compositions and methods for oxidative dyeing of hair.

2. The Related Art

When oxidation dyes of the type comprising primary intermediates and couplers are used in the dyeing of human hair, the procedure usually involves use of a two-part system. One part is an aqueous alkaline composition in liquid, gel or cream form containing oxidation dye precursors and an alkalizing agent. The other part is a developer usually containing an oxidizing agent such as hydrogen peroxide. Immediately prior to use, the two components are mixed together and applied to the hair.

A variety of factors can adversely influence the resultant intensity of coloration. Some of these include instability of the formulations during storage, slow diffusion of dye precursors from the dye mixture onto the hair fibers and poor rinsability of colorant subsequent to application.

U.S. Pat. No. 6,010,541 (de la Mettrie et al.) reports that nonionic amphiphilic polymers stabilize oxidation dye compositions against losing some of their gelled nature. Undesirable running of the composition is avoided resulting in a better localized application on the hair. More intense and luminous shades are thereby achievable.

U.S. Pat. No. 6,569,413 B1 (Hessefort et al.) discloses hair fixative compositions containing anionic polymers. These polymers can be formed from 2-acrylamido-2-methyl-1-propane sulfonic acid or salts thereof in combination with anionic or nonionic monomers. The declared benefit is a better balance between conflicting requirements of good curl retention at high humidity balanced by rapid and complete removal from the hair when rinsed with water.

The present invention is focused upon a method and composition to enhance the speed and quality of coloring hair through improvement of product stability.

### SUMMARY OF THE INVENTION

A method of coloring hair is provided which includes:

(a) applying to the hair a composition which includes;

(i) a colorant formula including from about 0.01 to about 10% by weight of the composition of an oxidation dye precursor in a cosmetically acceptable carrier; and

(ii) a developer formula including from about 0.01 to about 10% by weight of the composition of an alkoxylated taurate copolymer and from about 0.1 to about 10% by weight of the composition of hydrogen peroxide in a cosmetically acceptable carrier;

wherein the colorant and the developer formulas are applied to the hair separately or together in a relative weight ratio of 10:1 to 1:10; and

(b) removing the hair coloring composition from the hair.

Furthermore, there is provided a composition for dyeing hair which includes:

(i) from about 0.01 to about 100% by weight of the composition of an alkoxylated taurate copolymer;

(ii) from about 0.1 to about 100% by weight of the composition of hydrogen peroxide; and

(iii) a cosmetically acceptable carrier.

### DETAILED DESCRIPTION OF THE INVENTION

Now it has been found that alkoxylated taurate copolymers when incorporated with hydrogen peroxide in a developer formula used with an oxidation dye precursor compound can achieve improved stability thereby enhancing hair coloration. Alkoxylated taurate copolymers can at least partially be of anionic, nonionic or cationic charge. They may be crosslinked or non-crosslinked. The copolymer will include monomer units of 2-acrylamido-2-methylpropanesulphonic acid copolymerized either randomly or in block with a vinyl monomer. The taurate representing a polymerized unit of 2-acrylamido-2-methylpropanesulphonic acid may be neutralized from 0 to 100%, preferably from about 50 to about 90% and optimally from about 85 to about 99% of the available acid groups.

Most preferred are hydrophobically modified copolymers of acrylamidopropane sulphonic acid or salt and methacrylate esters of ethoxylated or propoxylated fatty alcohols.

Representative of the preferred alkoxylated taurate copolymers are those having the structure I.

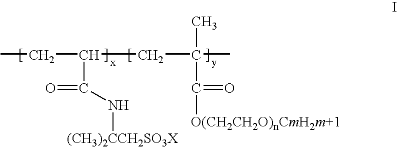

wherein X is selected from the group consisting of hydrogen, alkali metal, alkaline earth metal, ammonium, C₁-C₂₀ alkyl ammonium and combinations thereof; x and y may each range from 5 to 200; m may range from 12 to 24; and n may range from 5 to 50.

Particularly preferred as the alkoxylated taurate copolymer is the material known by its CTFA name as ammonium acryloyldi methyltaurates/beheneth-25 methacrylate copolymer, commercially available from Clariant Corporation as Aristoflex® HMB.

Amounts of the alkoxylated taurate copolymer may range from about 0.01 to about 100%, preferably from about 0.01 to about 5%, optimally from about 0.2 to about 2% by weight of the composition.

A source of hydrogen peroxide will be incorporated in the developer formula. Most suitable is aqueous hydrogen peroxide but precursors that can generate hydrogen peroxide may also be employed. The precursors include urea peroxide, sodium perborate, sodium percarbonate, calcium peroxide and sodium peroxide. The concentration of hydrogen peroxide may range from about 1 % to about 50%, preferably from about 3% to about 30%, optimally from about 8% to about 20% by weight of the composition.

A cosmetically acceptable carrier will be utilized with the developer 1 0 formula. Suitable cosmetic carriers include water, alcohols, hydrocarbons and combinations thereof. Suitable alcohols include the C₁-C₄ lower alcohols and the C₂-C₄₀ polyols such as propylene glycol, polyethylene glycol, glycerine and combinations thereof. Hydrocarbons may include C₈-C₃₀ isoparaffins, C₃-C₇ hydrocarbons, polyalphaolefins and mixtures thereof. Amounts of the carrier may range from about 100% to about 99.9%, preferably from about 80% to about 99%, optimally from about 90% to about 95% by weight of the developer formula.

Normally the developer will have a low pH ranging from about 2 to about 6, preferably from 2.5 to 3.5. Buffering agents may often be employed within the developer to maintain a desired pH level. Amounts of the buffering agents may from range from about 0.001 to about 2%, preferably from about 0.01 to about 0.1% by weight of the developer formula. Phosphoric, hydrochloric, sulphonic and C₂-C₃₀ carboxylic acids and their salts are useful as buffering agents. Illustrative examples agents include tartaric acid, citric acid, acetic acid, lactic acid, ammonium sulphate, sodium dihydrogen phosphate/phosphoric acid, potassium chloride/hydrochloric acid, potassium dihydrogen phthalate/hydrochloric acid, sodium citrate/hydrochloric acid, potassium dihydrogen citrate/hydrochloric acid, sodium tartarate/tartaric acid, sodium lactate/lactic acid, sodium acetate/acetic acid, disodium hydrogen phosphate/citric acid and sodium chloride/glycine/hydrochloric acid and mixtures thereof. Most preferred is phosphoric acid.

Surfactants may be present in the developer formula at levels from about 0.01 to about 20%, preferably the level of surfactant is minimized. In a preferred embodiment, the amount of surfactant may be less than 5%, preferably less than 1%, and more preferably less than 0.1% by weight of the developer formula.

The colorant formula according to the present invention may contain dye and coupler materials forming an oxidation dye precursor. Colors are produced by the reaction of the oxidation dye precursor with the oxidizing agent, such as hydrogen peroxide.

Suitable dye materials (sometimes referred to as developer compounds) include 1,4-diamino-benzene (p-phenylenediamine); 1,4-diamino-2-methyl-benzene (p-toluylenediamine); 1,4-diamino-2,6-dimethyl-benzene; 1,4-diamino-3,5-diethyl-benzene; 1,4-diamino-2,5-dimethyl-benzene; 1,4-diamino-2,3-dimethylbenzene; 2-chloro-1,4-diaminobenzene; 1,4-diamino-2-(thiophen-2-yl)benzene; 1,4-diamino-2-(thiophen-3-yl)benzene; 1,4diamino-2-(pyridin-3-yl)benzene; 2,5-diaminobiphenyl; 1,4-diamino-2-methoxymethyl-benzene; 1,4-diamino-2-aminomethylbenzene; 1,4-diamino-2-hydroxymethyl-benzene; 1,4-diamino-2-(2-hydroxyethoxy)benzene; 2-(2-(acetylamino)ethoxy)-1,4-diaminobenzene; 4-phenylamino-aniline; 4-dimethylamino-aniline; 4-diethylamino-aniline; 4-dipropylamino-aniline; 4[ethyl(2-hydroxyethyl)amino]-aniline; 4-[di(2-hydroxyethyl)amino]-aniline; 4-[di(2-hydroxyethyl)amino]-2-methyl-aniline; 4-[(2-methoxyethyl)amino]-aniline; 4-[(3-hydroxyropyl)amino]-aniline; 4-[(2,3-dihydroxypropyl)amino-aniline; 1,4-diamino-2-(2-hydroxyethyl)-benzene; 1,4-diamino-2-(1-methylethyl)-benzene; 1,3-bis[(4-aminophenyl)(2-hydroxyethyl)amino]-2-propanol; 1,4bis[(4-aminophenyl)amino]-butane; 1,8-bis(2,5-diaminophenoxy)-3,6-dioxaoctane; 4-amino-phenol; 4-amino-3-methyl-phenol; 4-amino-3-(hydroxymethyl)-phenol; 4-amino-3-fluoro-phenol; 4methylamino-phenol; 4-amino-2-(aminomethyl)-phenol; 4-amino-2-(hydroxymethyl)-phenol; 4-amino-2-fluorophenol; 4-amino-2-[(2-hydroxyethyl)-amino]methylphenol; 4-amino-2-methyl-phenol; 4-amino-2-(methoxymethyl)-phenol; 4-amino-2-(2-hydroxyethyl)-phenol; 5-amino-salicylic acid; 25-diamino-pyridine; 2,4,5,6-tetraamino-pyrimidine; 2,5,6-diamino-4(1H)-pyrimidone; 4,5-diamino-1-1(2-hydroxyethyl)-1H-pyrazole; 4, 5-diamino-1-(1 -methylethyl)-1H-pyrazole; 4,5-diamino-1-[(4-methylphenyl)methyl]-1H-pyrazole; 1-[(4-chlorophenyl)methyl]-4,5-diamino-1H-pyrazole; 4,5-diamino-1-methyl-1H-pyrazole; 2-aminophenol; 2-amino-6-methyl phenol and 2-amino-5-methylphenol.

The dye materials may be used individually or together with each other in a mixture.

Suitable coupler materials include N-(3-dimethylamino-phenyl)-urea, 2,6-diamino-pyridine; 2-amino-4-[(2-hydroxyethyl)amino]anisole; 2,4-diamino-1,4-fluoro-5-methylbenzene; 2,4-diamino-1-methoxy-5-methylbenzene 2,4-diamino-1-ethoxy-5-methyl-benzene; 2,4-diamino-1-(2-hydroxyethoxy)-5-methylbenzene; 2,4-di[(2-hydroxyethyl)amino]-1,5-dimethoxybenzene; 2,3-diamino-6-methoxy-pyridine; 3-amino-6-methoxy-2-(methylamino)pyridine; 2,6-diamino-3,5-dimethoxypyridine; 3,5-diamino-2,6-dimethoxy-pyridine; 1,3-diaminobenzene; 2,4-diamino-1-(2-hydroxyethoxy)benzene; 1,3-diamino-4-(2,3-hydroxpropoxy)benzene; 2,4-diamino-1,5-di[(2-hydroxyethoxy)-benzene; 1-(2-aminoethoxy)-2,4-diaminobenzene; 2-amino-1-(2-hydroxyethoxy)-4-methylaminobenzene; 2,4-diaminophenoxyacetic acid ester; 3-[di(2-hydroxyethyl)aminolaniline; 4-amino-2-di[(2-hydroxyethyl)amino]-1-ethoxy-benzene; 5-methyl-2-(1-methyethyl)phenol; 3-[(2-hydroxyethyl)amino]aniline; 3-[(2-aminoethyl)amino]aniline; 1,3-di(2,4-diaminophenoxy)propane; di(2,4-diaminophenoxy)methane; 1,3-diamino-2,4-dimethoxybenzene; 2,6-bis(2-hydroxyethyl)aminotoluene; 4-hydroxyindole; 3-dimethylaminophenol; 3-diethylaminophenol; 5-amino-2-methylphenol; 5-aminofluoro-2-methyl-phenol; 5-amino-4-methoxy-2-methylphenol; 5-amino4-ethoxy-2-methylphenol; 3-amino-2,4-dichlorophenol; 5-amino-2,4-dichlorophenol; 3-amino-2-methyl-phenol; 3-amino-2-chloro-6-methylphenol; 3-aminophenol; 2-[(3-hydroxyphenol)-amino]acetamide; 5-[(2-hydroxyethyl)amino]-4-methoxy-2-methyl phenol; 5-[(2-hydroxyethyl)amino]-2-methylphenol; 3-[(2-hydroxyethyl)amino]-phenol; 3-[(2-methoxyethyl)amino]-phenol; 5-amino-2-ethyl-phenol; 5-amino-2-methoxyphenol; 2-(4-amino-2-hydroxyphenoxy)ethanol; 5-[(3-hydroxypropyl)amino]-2-methylphenol; 3-[(2,3-dihydroxypropyl)amino]-2-methylphenol; 3-[(2-hydroxyethyl)amino]-2methylphenol; 2-amino-3-hydroxypyridine; 5-amino-4-chloro-2-methylphenol; 1 -naphthol; 2-methyl-1-naphthol; 1,5-dihydroxynaphthalene; 1,7-dihydroxy-naphthalene; 2,3-dihydroxynaphthalene; 2,7-dihydroxy-naphthalene; 2-methyl-1-naphthol-acetate; 1,3-dihydroxybenzene; 1-chloro-2,4-dihydroxy-benzene; 2-chloro-1,3-dihydroxybenzene; 1,2-dichloro-2,4-dihydroxy-4-methylbenzene; 1,5-dichloro-2,4-dihydroxy-benzene; 1,3-dihydroxy-2-ethyl-benzene; 3,4-methylenedioxy-phenol; 3,4-methylenedioxy-aniline; 6-bromo-1-hydroxy-3,4methylenedioxybenzene; 3,4-diaminobenzoic acid; 3,4-dihydroxy-6-hydroxy-1,4(2H)benzoxazine; 6-amino-3,4-dihydro-1,4(2 H)-benzoxazine; 3-methyl-1-phenyl-5-pyrazolone; 5,6-dihydroxyindole; 5,6-dihydroxyindolene; 5-hydroxyindole; 6-hydroxyindole; 7-hydroxyindole and 2,4-indolendione. The suitable coupler materials can be used individually, or together with each other in a mixture.

Self-coupling oxidation dye precursors include 2-amino-5-methylphenol; 2-amino-6-methylphenol; 2-amino-5-ethoxyphenol and 2-propyl-amino-5-aminopyridine.

The oxidation dye precursor may be present in amounts from about 0.01 to about 10%, preferably from about 0.2 to about 6% by weight of the combined composition.

The colorant formula may contain one or more alkalizing agents present in an amount from about 0.5 to 5% by weight of the combined composition. The term “alkalizing agent” means an ingredient that is capable of imparting alkalinity (e.g. a pH of greater than 7) to the colorant formula. Suitable alkalizing agents include ammonium and metal hydroxides, alkanolamines, sodium silicates, ammonium and metal carbonates, sodium metasilicates, and mixtures thereof. Examples of metal hydroxides are sodium, potassium, lithium, calcium and magnesium hydroxide. A particularly preferred alkaline earth metal hydroxide is sodium hydroxide. Alkanolamines include mono-, di-, and trialkanolamines such as monoethanolamine (MEA), diethanolamine (DEA), triethanolamine (TEA), 2-aminobutanol, aminoethyl propanediol, aminomethyl propanediol, bis-hydroxyethyltromethamine, diethyl ethanolamine, diisopropanolamine, dimethylamino methyl propanol, dimethyl MEA, isopropanolamine, methylethanolamine, mixed isopropanolamines, triisopropanolamine, tromethamine, and mixtures

thereof. A particularly preferred alkanolamine is MEA.

The colorant formula may contain one or more fatty acids, and if so suggested ranges are about 0.001-15%, preferably 0.005-10%, most preferably 0.01-8% by weight of the total composition. If fatty acids are present they will react with the alkalizing agent to form soap in situ, which provides a more shampoo-like character to the aqueous hair color composition once it is applied to hair. Suitable fatty acids include oleic acid, stearic acid, myristic acid, and linoleic acid. Particularly preferred is oleic acid.

The colorant formula may comprise one or more conditioners that exert a conditioning effect on hair. A variety of conditioners are suitable including cationic polymers, oily conditioning agents, silicones, fatty alcohols, proteins, and mixtures thereof. A combined total weight of conditioners may range from about 0.1 to about 25%, preferably 0.5 to 20%, more preferably 1 to 15% by weight of the combined composition.

Illustrative cationic polymers are quaternary derivatives of cellulose ethers or guar derivatives, copolymers of vinylpyrrolidone, polymers of dimethyldiallyl ammonium chloride, acrylic or methacrylic polymers and quaternary ammonium polymers.

Examples of quaternary derivatives of cellulose ethers are polymers sold under the trademark JR-125, JR-400 and JR-30M. Suitable guar derivatives include guar hydroxypropyl trimonium chloride.

Oily conditioning agents are liquid at room temperature and may comprise esters and hydrocarbons. Examples include sunflowerseed oil, soybean oil, C₁₁₋₁₃ isoparafin and mineral oil. Amounts may range from about 0.001 to about 20%, preferably from 0.005 to 5%, more preferably from 0.01 to 100% by weight of the combined composition.

Silicone hair conditioning agents include volatile or nonvolatile nonionic silicone fluids, silicone resins, and silicone semi-solids or solids.

Volatile silicones are linear or cyclic silicones having a measurable vapor pressure, which is defined as a vapor pressure of at least 2 mm at 20° C. Examples include cyclomethicones such as sold by Dow Corning under the DC 245, DC 244 and DC 344 designations.

Nonvolatile silicones may also be utilized. These can include polyalkyl siloxanes, polyaryl siloxanes, polyalkyl aryl siloxanes, polyether siloxane copolymers, amine-functional silicones and mixtures thereof.

Amounts of the silicone may range from about 0.001 to about 20%, preferably from about 0.005 to about 5, more preferably from about 0.01 to 4% by weight of the combined composition.

The colorant formula may comprise one or more surfactants that assist in maintaining the emulsion form and aid in the foaming capability of the composition. Suitable surfactants include anionic, nonionic and amphoteric surfactants.

Amounts of the nonionic surfactant may range from about 0.01 to about 20%, preferably from about 0.05 to 10%, more preferably from about 0.1 to 5% by weight of the total combined composition. Suitable nonionic surfactants include alkoxylated alcohols or ethers, alkoxylated carboxylic acids, sorbitan derivatives. Examples include steareth-21, oleth-20, polysorbate and sorbitan oleate.

If desired the colorant formula may contain one or more anionic surfactants. Amounts may range about 0.1 to about 25%, preferably from 0.5 to 20%, more preferably from 1 to 15% by weight of the total combined composition. Suitable anionic surfactants include alkyl and alkyl ether sulfates generally having the formula ROSO₃M and RO(C₂H₄O)_{X}SO₃M wherein R is alkyl or alkenyl of from about 10 to 20 carbon atoms, x is 1 to about 10 and M is a water soluble cation such as ammonium, sodium, potassium, or triethanolamine cation.

It may be desirable to include one or more solvents in the colorant formula. Solvents assist in solubilizing the dye precursors in addition to other ingredients in the composition. The solvent when present may range from about 0.01 to 20%, preferably from 0.05 to 10%, more preferably from 0.1 to 6% by weight of the total combined composition. Suitable solvents include C₂₋₄ alkanols such as ethanol, isopropanol, and alkoxydiglycols such as ethoxydiglycol.

The colorant formula may contain one or more chelating agents that are capable of chelating the metal ions found in water. If water contains too many extraneous metal ions they can interfere with the coloration process. Amounts of the chelating agent may range from about 0.001 to about 5%, preferably from about 0.005 to 4%, more preferably from 0.01 to 2% by weight of the total combined composition. Preferred chelating agents are EDTA, HEDTA, and sodium or potassium salts thereof.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

Representative of the present invention are the following colorant and developer formulas illustrated in Tables I and II.

TABLE I
Colorant Formula

| Component | Weight % |
|---|---|
| Oleic Acid | 6.00 |
| C12-15 Pareth-9 | 1.00 |
| C12-15 Pareth-3 | 2.00 |
| PEG-2 Soyamine | 4.00 |
| Propylene Glycol | 7.00 |
| Isopropanol | 13.00 |
| EDTA | 0.10 |
| Sodium Sulfite | 0.80 |
| Sodium Isoascorbate | 0.15 |
| P-Aminophenol | 0.75 |
| P-Amino-O-Cresol | 0.80 |
| NH₄OH (28% Active) | 6.00 |
| Water | To 100 |

TABLE II
Developer Formula

| Component | Weight % |
|---|---|
| Aristoflex ® HMB | 0.80 |
| Hydrogen Peroxide (50% Active) | 12.00 |
| Phosphoric Acid (85% Active) | 0.05 |
| Citric Acid (50% Active) | 0.10 |
| Water | To 100 |

### EXAMPLE 2

Another Example of the present invention are the colorant and developer formulas outlined under Table III and IV.

TABLE III
Colorant Formula

| Component | Weight % |
|---|---|
| Sodium Lauryl Ether Sulphate (28% Active) | 3.00 |
| 2,5-Diaminotoluene Sulphate | 2.80 |
| Resorcinol | 1.00 |
| m-Aminophenol | 0.40 |
| 2-Amino-4-(2-Hydroxyethanolamino) Anisole Sulphate | 0.20 |
| Ascorbic Acid | 0.30 |
| EDTA | 0.10 |
| Ammonia (25% Active in water) | 12.20 |
| Ethanol | 2.00 |
| Water | To 100 |

TABLE IV
Developer Formula

| Component | Weight % |
|---|---|
| Cetearyl Alcohol | 10.00 |
| Aristoflex ® HMB | 4.00 |
| Hydrogen Peroxide (35% Active) | 17.00 |
| Perfume | 0.30 |
| Water | To 100 |

### EXAMPLE 3

A still further illustration of formulas according to the present invention are the colorant and developer formulas illustrated in Tables V and VI.

TABLE V
Colorant Formula

| Component | Weight % |
|---|---|
| Cocoamidopropyl Betaine | 5.00 |
| Ammonium Carbonate | 1.20 |
| Sodium Citrate | 0.40 |
| Stearalkonium Chloride | 2.00 |
| p-Phenylenediamine | 0.50 |
| Resorcinol | 0.20 |
| EDTA | 0.10 |
| Sodium Sulfite | 0.10 |
| Perfume | 0.10 |
| Water | To 100 |

TABLE VI
Developer Formula

| Component | Weight % |
|---|---|
| Hydrogen Peroxide | 6.00 |
| Aristoflex ® HMB | 1.00 |
| EDTA | 0.10 |
| Phosphoric Acid | 0.10 |
| Water | To 100 |

### EXAMPLE 4

This Example illustrates the stability imparted by various copolymers to the developer formula. Table VII demonstrates that Aristoflexe HMB imparts an effective stability to a hydrogen peroxide system. By contrast, an acrylate/beheneth-25 methacrylate copolymer (evaluated on an identical solids basis) separated after 3 weeks in an accelerated stability test.

TABLE VII

| | Sample | | |
|---|---|---|---|
| Component | A | B | C |
| AristofIex ® HMB | 0.5 | — | — |
| Aculyn 28* | — | 2.5 | — |
| Aculyn 33* | — | — | 1.78 |
| Hydrogen Peroxide | 12 | 12 | 12 |
| (50% Active) | | | |
| Phosphoric Acid (85% | 0.05 | 0.05 | 0.05 |
| Active) | | | |
| Water | To 100 | To 100 | To 100 |
| Citric Acid (50% | Adjust to pH 3 | Adjust to pH 3 | Adjust to pH 3 |
| Active) | | | |
| Stability at 45° C. | Stable | Separated after | Separated after |
| | | 3 weeks | 3 weeks |

*Aculyn 28 is an anionic acrylate/beheneth-25 methacrylate copolymer with 20% solids made by Rohm and Haas. Aculyn 33 is an anionic acrylate/methacrylate copolymer with 28% solids.

What is claimed is:

1. A method of coloring hair comprising:
(a) applying to the hair a composition comprising;
(i) a colorant formula including from about 0.01 to about 10% by weight of the composition of an oxidation dye precursor in a cosmetically acceptable carrier; and
(ii) a developer formula including from about 0.01 to about 10% by weight of the composition of an alkoxylated taurate copolymer and from about 0.1 to about 10% by weight of the composition of hydrogen peroxide in a cosmetically acceptable carrier;
wherein the colorant and the developer formulas are applied to the hair separately or together in a relative weight ratio of 10:1 to 1:10; and
(b) removing the hair coloring composition from the hair.

2. The method according to claim 1 wherein the alkoxylated taurate copolymer is a hydrophobically modified copolymer of acrylamidopropane sulphonic acid or salt and methacrylate esters of ethoxylated or propoxylated fatty alcohols.

3. The method according to claim 2 wherein the alkoxylated taurate copolymer has the structure I. 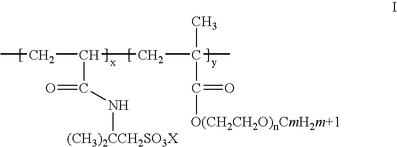
wherein X is selected from the group consisting of hydrogen, alkali metal, alkaline earth metal, ammonium, C₁-C₂₀ alkyl ammonium and combinations thereof; x and y may each range from 5 to 200; m may range from 12 to 24; and n may range from 5 to 50.

4. The method according to claim 1 wherein the alkoxylated taurate copolymer is a salt of acryloyldimethyltaurates/beheneth-25 methacrylate copolymer.

5. The method according to claim 4 wherein the amount of alkoxylated taurate copolymer ranges from about 0.2 to about 2% by weight of the composition.

6. A composition for dyeing hair comprising:
(i) from about 0.01 to about 10% by weight of the composition of an alkoxylated taurate copolymer;
(ii) from about 0.1 to about 10% by weight of the composition of hydrogen peroxide; and
(iii) a cosmetically acceptable carrier.

7. The composition according to claim 6 wherein the alkoxylated taurate copolymer is a hydrophobically modified copolymer of acrylamidopropane sulphonic acid or salt and methacrylate esters of ethoxylated or propoxylated fatty alcohols.

8. The composition according to claim 7 wherein the alkoxylated taurate has the structure I. 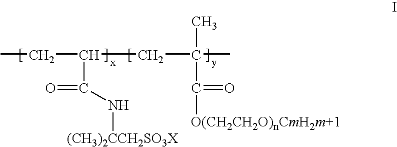
wherein X is selected from the group consisting of hydrogen, alkali metal, alkaline earth metal, ammonium, C₁-C₂₀ alkyl ammonium and combinations thereof; x and y may each range from 5 to 200; m may range from 12 to 24; and n may range from 5 to 50.

9. The composition according to claim 6 wherein the alkoxylated taurate copolymer is a salt of acryloyldimethyltaurates/beheneth-25 methacrylate copolymer.
